(19) United States
(12) Patent Application Publication
(10) Pub. No.: US 2021/0198173 A1
(43) Pub. Date: Jul. 1, 2021

(54) OXIDATION OF METHYL-SUBSTITUTED BIPHENYL COMPOUNDS

(71) Applicant: ExxonMobil Chemical Patents Inc., Baytown, TX (US)

(72) Inventors: Jihad M. Dakka, Whitehouse Station, NJ (US); Bryan A. Patel, Jersey City, NJ (US); Michael Salciccioli, Ann Arbor, MI (US); Stephen Zushma, Clinton, NJ (US)

(21) Appl. No.: 17/180,445

(22) Filed: Feb. 19, 2021

Related U.S. Application Data
(62) Division of application No. 16/071,856, filed on Jul. 20, 2018.
(60) Provisional application No. 62/320,014, filed on Apr. 8, 2016.

(51) Int. Cl. C07C 51/265; B01J 21/04; B01J 21/08; B01J 23/44; B01J 23/62; B01J 29/74; C07C 2/84; C07C 5/48; C07C 51/353; C07C 51/43; C07C 51/44

(52) U.S. Cl. CPC C07C 51/265; C07C 2529/70; B01J 21/08; B01J 23/44; B01J 23/626; B01J 29/74; C07C 2/84; C07C 5/48; C07C 51/353; C07C 51/43; C07C 51/44; C07B 2200/07; C07C 2521/04; C07C 2521/08; C07C 2523/14; C07C 2523/42; C07C 2523/44; C07C 2529/18; C07C 2529/22; B01J 21/04

(57) ABSTRACT

A process for oxidizing methyl-substituted biphenyl compounds comprises contacting a mixture comprising isomers of at least one methyl-substituted biphenyl compound with a source of oxygen, wherein the mixture comprises at least 20 wt % of isomer(s) having a methyl group at a 2-position or a 3-position on at least one benzene ring and at least 50 wt % of isomer(s) having a methyl group at a 4-position on at least one benzene ring, wherein said percentages are based on the total weight of the at least one methylbiphenyl compound in the mixture.

### CROSS REFERENCE TO RELATED APPLICATION

This invention claims priority to and the benefit of U.S. Ser. No. 62/320,014, filed Apr. 8, 2016.

### FIELD OF THE INVENTION

This disclosure relates to the oxidation of methyl-substituted biphenyl compounds.

### BACKGROUND OF THE INVENTION

Methyl-substituted biphenyl (MBP) compounds, and especially dimethylbiphenyl (DMBP) compounds, are useful intermediates in the production of a variety of commercially valuable products, including polyesters and plasticizers for PVC and other polymer compositions. For example, methyl-substituted biphenyl compounds can be converted to ester plasticizers by a process comprising oxidation to produce the corresponding carboxylic acid followed by esterification with a long chain alcohol. In addition, diphenyl dicarboxylic acids are potential precursors, either alone or as a modifier for polyethylene terephthalate (PET), in the production of polyester fibers, engineering plastics, liquid crystal polymers for electronic and mechanical devices, and films with high heat resistance and strength.

As disclosed in our co-pending U.S. Ser. No. 14/201,287 and 14/201,224, both filed Mar. 7, 2014, DMBP compounds may be produced by hydroalkylation of toluene followed by dehydrogenation of the resulting (methylcyclohexyl)toluene (MCHT). The product comprises a mixture of all six DMBP isomers, namely 2,2′, 2,3′, 2,4′, 3,3′, 3,4′, and 4,4′ DMBP, in which the 3,4′ DMBP isomer is usually the most abundant. The entire disclosures of U.S. Ser. No. 14/201,287 and 14/201,224, are fully incorporated herein by reference in their entirety.

Alternative routes to MBP compounds via benzene are described in co-pending U.S. Ser. No. 14/164,889, filed Jan. 27, 2014, in which the benzene is initially converted to biphenyl, either by oxidative coupling or by hydroalkylation to cyclohexyl benzene (CHB) followed by dehydrogenation of the CHB, and then the biphenyl is alkylated with methanol. The alkylated product comprises a mixture of MBP isomers.

For certain uses, it is important to control the level of specific isomers in MPB isomer mixtures. Thus, for example, diphenate esters derived from 3,3′-DMPB often exhibit improved low temperature flex properties when used as plasticizers, whereas diphenate esters derived from 2,2′-DMPB are typically too volatile for use as plasticizers.

One problem associated with the commercial exploitation of methyl-substituted biphenyl compounds as a route to products, such as plasticizers and polyesters, is the oxidation step to produce the acid intermediate. Thus, it has been found that the oxidation rate with known oxidation catalysts, such as Co and/or Mn, varies significantly between the different isomers, with the 4-isomers generally exhibiting the highest oxidation rate. For example, the hierarchy between the oxidation rates for DMPB isomers is as follows: 4,4′>3,4′>2,4′>3,3′>2,3′. There is, therefore, interest in developing an improved oxidation regime in which the oxidation rate of the less active isomers, such as the 3,3′ and the 2,X′ (where X′ is 3′ or 4′) DMPB isomers, is increased.

### SUMMARY OF THE INVENTION

According to the present invention, it has now been found that the oxidation of less reactive methyl-substituted biphenyl compounds, that is those containing one or more methyl groups at the 2- or 3-position, can be enhanced by the addition of one or more isomers containing methyl groups at the 4-position. While the reason for this result is not fully understood, and without wishing to be bound by theory, it is believed that the isomers with methyl groups at the 4-position are highly active in initiating the generation of free radicals and in maintaining a high steady state concentration of such radicals even in the absence of external radical initiators, such as bromine compounds.

In one aspect, the invention resides in a process for oxidizing methyl-substituted biphenyl compounds, the process comprising:
(a1) providing a mixture comprising isomers of at least one methyl-substituted biphenyl compound, wherein the mixture comprises at least 20 wt % of isomer(s) having a methyl group at a 2-position or a 3-position on at least one benzene ring and at least 50 wt % of isomer(s) having a methyl group at a 4-position on at least one benzene ring, wherein said percentages are based on the total weight of the at least one methylbiphenyl compound in the mixture; and
(b1) contacting the mixture with a source of oxygen.

In a further aspect, the invention resides in a mixture comprising from 20 to 50 wt % of at least one dimethylbiphenyl isomer selected from the group consisting of 2,3′ and 3,3′ dimethylbiphenyl isomers and from 50 to 80 wt % of one or more 4,Y′ (where Y′ is 3′ or 4′) dimethylbiphenyl isomers, wherein said percentages are based on the total weight of all dimethylbiphenyl isomers in the mixture.

In yet a further aspect, the invention resides in a process for producing methyl biphenyl carboxylic acid and/or biphenyl dicarboxylic acid, the process comprising:
(a2) contacting a feed comprising toluene with hydrogen in the presence of a hydroalkylation catalyst under conditions effective to produce a hydroalkylation reaction product comprising (methylcyclohexyl)toluenes;
(b2) dehydrogenating at least part of the hydroalkylation reaction product in the presence of a dehydrogenation catalyst under conditions effective to produce a dehydrogenation reaction product comprising dimethylbiphenyl isomers;
(c2) adjusting the composition of at least part of the dehydrogenation reaction product to produce a mixture comprising from 20 to 50 wt % of at least one dimethylbiphenyl isomer selected from the group consisting of 2,3′ and 3,3′ dimethylbiphenyl isomers and from 50 to 80 wt % of one or more 4,Y′ (where Y′ is 3′ or 4′) dimethylbiphenyl isomers, wherein said percentages are based on the total weight of all dimethylbiphenyl isomers in the mixture; and
(d2) oxidizing the mixture produced in (c1).

In another aspect, the invention resides in a process for producing methyl biphenyl carboxylic acid and/or biphenyl dicarboxylic acid, the process comprising:
(a3) contacting a feed comprising benzene with hydrogen in the presence of a hydroalkylation catalyst under conditions effective to produce a hydroalkylation reaction product comprising cyclohexylbenzenes;
(b3) dehydrogenating at least part of the hydroalkylation reaction product in the presence of a dehydrogenation catalyst under conditions effective to produce a dehydrogenation reaction product comprising biphenyl;
(c3) reacting at least part of the dehydrogenation reaction product with a methylating agent in the presence of an alkylation catalyst under conditions effective to produce a methylation reaction product comprising methyl-substituted biphenyl compounds;
(d3) adjusting the composition of at least part of the methylation reaction product to produce a mixture comprising isomers of at least one methyl-substituted biphenyl compound, wherein the mixture comprises at least 20 wt % of isomer(s) having a methyl group at a 2-position or a 3-position on at least one benzene ring and at least 50 wt % of isomer(s) having a methyl group at a 4-position on at least one benzene ring, wherein said percentages are based on the total weight of the at least one methylbiphenyl compound in the mixture; and
(e3) oxidizing the mixture produced in (d3).

In still another aspect, the invention resides in a process for producing methyl biphenyl carboxylic acid and/or biphenyl dicarboxylic acid, the process comprising:
(a4) oxidizing a feed comprising benzene in the presence of an oxidative coupling catalyst under conditions effective to produce an oxidation reaction product comprising biphenyl;
(b4) reacting at least part of the oxidation reaction product with a methylating agent in the presence of an alkylation catalyst under conditions effective to produce a methylation reaction product comprising methyl-substituted biphenyl compounds;
(c4) adjusting the composition of at least part of the methylation reaction product to produce a mixture comprising isomers of at least one methyl-substituted biphenyl compound, wherein the mixture comprises at least 20 wt % of isomer(s) having a methyl group at a 2-position or a 3-position on at least one benzene ring and at least 50 wt % of isomer(s) having a methyl group at a 4-position on at least one benzene ring, wherein said percentages are based on the total weight of the at least one methylbiphenyl compound in the mixture; and
(d4) oxidizing the mixture produced in (c4).

### BRIEF DESCRIPTION OF THE DRAWINGS

FIG. 1 is a graph showing the calculated equilibrium distribution of dimethylbiphenyl isomers over a temperature range from 20 to 750° C.

FIG. 2 is a gas chromatograph (GC) mass spectrum of the product obtained using the Pd/MCM-49 catalyst of Example 1 in the toluene hydroalkylation process of Example 3.

FIG. 3 is a gas chromatograph (GC) mass spectrum of the product obtained using the Pd/beta catalyst of Example 2 in the toluene hydroalkylation process of Example 3.

FIG. 4 is a bar graph comparing the products obtained in the dehydrogenation process of Example 5.

FIG. 5 is a graph of isomer conversion against total DMPB conversion for oxidation of the DMPB isomer mixture of Example 6.

FIG. 6 is a graph comparing conversion against time on stream for oxidation of 3,3′-DMPB in the isomer mixture used in Example 7 with the pure 3,3′-DMPB used in Example 7.

FIG. 7 is a graph comparing monocarboxylic acid selectivity against feed conversion for oxidation of 3,3′-DMPB in the isomer mixture used in Example 7 with the pure 3,3′-DMPB used in Example 7.

FIG. 8 is a graph comparing dicarboxylic acid selectivity against feed conversion for oxidation of 3,3′-DMPB in the isomer mixture used in Example 7 with the pure 3,3′-DMPB used in Example 7.

FIG. 9 is a graph comparing conversion against time on stream for oxidation of 2,3′-DMPB in the isomer mixture used in Example 7 with the pure 2,3′-DMPB used in Example 8.

FIG. 10 is a graph of total DMPB conversion against time on stream for oxidation of the DMPB isomer mixture of Example 9 with and without spiking with 1 wt % (methylcyclohexyl)toluene and 100 ppmw fluorene.

FIG. 11 is a graph comparing monocarboxylic acid selectivity against feed conversion for oxidation of the DMPB isomer mixture of Example 9 with and without spiking with 1 wt % (methylcyclohexyl)toluene and 100 ppmw fluorene.

FIG. 12 is a graph comparing dicarboxylic acid selectivity against feed conversion for oxidation of the DMPB isomer mixture of Example 9 with and without spiking with 1 wt % (methylcyclohexyl)toluene and 100 ppmw fluorene.

### DETAILED DESCRIPTION OF THE INVENTION

Described herein is a process of oxidizing methyl-substituted biphenyl compounds in which the rate of oxidation of isomer(s) having a methyl group at a 2-position or a 3-position on at least one benzene ring is enhanced by the presence in the oxidation reaction mixture of isomer(s) having a methyl group at a 4-position on at least one benzene ring. The process is applicable to any mixture of methyl-substituted biphenyl isomers having the required methyl group distribution including, for example, (a) mixtures of monomethyl-substituted biphenyl isomers in which each isomer has a single methyl substituent on one of the two benzene rings, (b) mixtures of dimethyl-substituted biphenyl isomers in which each isomer has a single methyl substituent on each of the two benzene rings, (c) mixtures of trimethyl-substituted biphenyl isomers in which each isomer has a single methyl substituent on one benzene ring and two methyl substituents on the other benzene ring, and (d) tetra (and higher) methyl-substituted biphenyl isomers in which each isomer has at least two methyl substituents on each benzene ring.

In one preferred embodiment of the invention, the process is directed to the oxidation of a mixture of dimethyl-substituted biphenyl (DMPB) isomers, of which the 3,3′, 3,4′ and 4,4′-isomers are shown below in formulas (I) to (III) respectively, whereas the 2,2′, 2,3′ and 2,4′-isomers are shown in formulas (IV) to (VI) respectively:

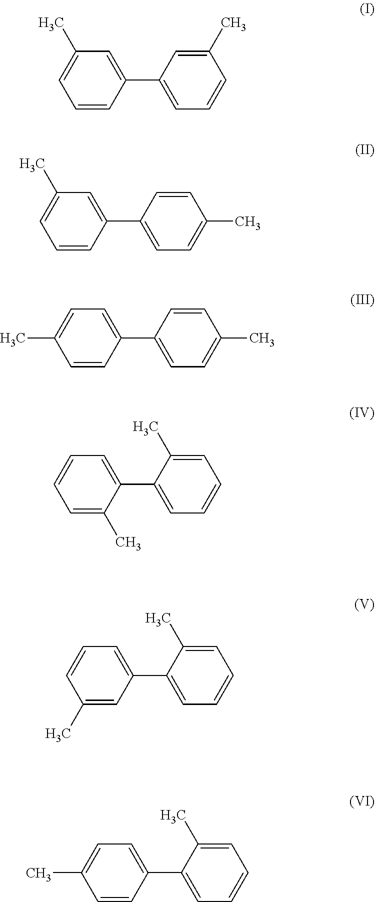

The present process is particularly applicable to a mixture of DMPB isomers comprising at least one 2,3′- or 3,3′-isomer and at least one 2,4′-, 3-4′, or 4,4′ isomer.

### Production of Methyl-Substituted Biphenyl Isomers

The method used to produce the mixture of methyl-substituted biphenyl isomers oxidized in accordance with the present process is not critical. However, suitable isomer mixtures may be produced via hydroalkylation of toluene, benzene, xylene, and mixtures thereof. For example, toluene may be converted to (methylcyclohexyl)toluenes over a hydroalkylation catalyst according to the following reaction.

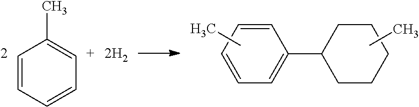

The catalyst employed in the hydroalkylation reaction is a bifunctional catalyst comprising a hydrogenation component and a solid acid alkylation component, typically a molecular sieve. The catalyst may also include a binder such as clay, alumina, silica, and/or metal oxides. The latter may be either naturally occurring or in the form of gelatinous precipitates or gels, including mixtures of silica and metal oxides. Naturally occurring clays, which can be used as a binder, include those of the montmorillonite and kaolin families, which families include the subbentonites and the kaolins commonly known as Dixie, McNamee, Ga., and Florida clays, or others in which the main mineral constituent is halloysite, kaolinite, dickite, nacrite, or anauxite. Such clays can be used in the raw state as originally mined or initially subjected to calcination, acid treatment, or chemical modification. Suitable metal oxide binders include silica, alumina, zirconia, titania, silica-alumina, silica-magnesia, silica-zirconia, silica-thoria, silica-beryllia, silica-titania, as well as ternary compositions, such as silica-alumina-thoria, silica-alumina-zirconia, silica-alumina-magnesia and silica-magnesia-zirconia.

Any known hydrogenation metal or compound thereof can be employed as the hydrogenation component of the hydroalkylation catalyst, although suitable metals include palladium, ruthenium, nickel, zinc, tin, cobalt, silver, gold, platinum, and compounds and mixtures thereof, with palladium being particularly advantageous. In certain embodiments, the amount of hydrogenation metal present in the catalyst is between about 0.05 and about 10 wt %, such as between about 0.1 and about 5 wt %, of the catalyst.

In one embodiment, the solid acid alkylation component comprises a large pore molecular sieve having a Constraint Index (as defined in U.S. Pat. No. 4,016,218) less than 2. Suitable large pore molecular sieves include zeolite beta, zeolite Y, Ultrastable Y (USY), Dealuminized Y (Deal Y), mordenite, ZSM-3, ZSM-4, ZSM-18, and ZSM-20. Zeolite ZSM-4 is described in U.S. Pat. No. 4,021,447. Zeolite ZSM-20 is described in U.S. Pat. No. 3,972,983. Zeolite Beta is described in U.S. Pat. No. 3,308,069 and US Re. 28,341. Low sodium Ultrastable Y molecular sieve (USY) is described in U.S. Pat. Nos. 3,293,192 and 3,449,070. Dealuminized Y zeolite (Deal Y) may be prepared by the method found in U.S. Pat. No. 3,442,795. Zeolite UHP-Y is described in U.S. Pat. No. 4,401,556. Mordenite is a naturally occurring material, but is also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104. Preferred large pore molecular sieves for use as the solid acid alkylation component of the hydroalkylation catalyst comprise molecular sieves of the BEA and FAU structure type.

In another, more preferred embodiment, the solid acid alkylation component comprises a molecular sieve of the MCM-22 family. The term “MCM-22 family material” (or “material of the MCM-22 family” or “molecular sieve of the MCM-22 family”), as used herein, includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Molecular sieves of MCM-22 family generally have an X-ray diffraction pattern including d-spacing maxima at 12.40.25, 6.90.15, 3.570.07 and 3.420.07 Angstrom. The X-ray diffraction data used to characterize the material are obtained by standard techniques using the K-alpha doublet of copper as the incident radiation and a diffractometer equipped with a scintillation counter and associated computer as the collection system. Molecular sieves of MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in EP 0 293 032), ITQ-1 (described in U.S. Pat. No. 6,077,498), ITQ-2 (described in WO 97/17290), MCM-36 (described in U.S. Pat. No. 5,250,277), MCM-49 (described in U.S. Pat. No. 5,236,575), MCM-56 (described in U.S. Pat. No. 5,362,697) and mixtures thereof.

In addition to the toluene and hydrogen, a diluent, which is substantially inert under hydroalkylation conditions, may be included in the feed to the hydroalkylation reaction. In certain embodiments, the diluent is a hydrocarbon, in which the desired cycloalkylaromatic product is soluble, such as a straight chain paraffinic hydrocarbon, a branched chain paraffinic hydrocarbon, and/or a cyclic paraffinic hydrocarbon. Examples of suitable diluents are decane and cyclohexane. Although the amount of diluent is not narrowly defined, desirably the diluent is added in an amount such that the weight ratio of the diluent to the aromatic compound is at least 1:100; for example at least 1:10, typically no more than 10:1, desirably no more than 4:1.

The hydroalkylation reaction can be conducted in a wide range of reactor configurations including fixed bed, slurry reactors, and/or catalytic distillation towers. In addition, the hydroalkylation reaction can be conducted in a single reaction zone or in a plurality of reaction zones, in which at least the hydrogen is introduced to the reaction in stages. Suitable reaction temperatures are between about 100° C. and about 400° C., such as between about 125° C. and about 250° C., while suitable reaction pressures are between about 100 and about 7000 kPa, such as between about 500 and about 5000 kPa. The molar ratio of hydrogen to aromatic feed, such as toluene, is typically from about 0.15:1 to about 15:1.

The major components of the hydroalkylation reaction effluent are (methylcyclohexyl)toluenes, residual toluene and fully saturated single ring by-products, such as methylcyclohexane. The residual toluene and light by-products can readily be removed from the reaction effluent by, for example, distillation. The residual toluene can then be recycled to the hydroalkylation reactor, while the saturated by-products can be dehydrogenated to produce additional recyclable feed.

The remainder of the hydroalkylation reaction effluent, composed mainly of (methylcyclohexyl)toluenes, is then dehydrogenated to convert the (methylcyclohexyl)toluenes to the corresponding methyl-substituted biphenyl compounds. The dehydrogenation is conveniently conducted at a temperature from about 200° C. to about 600° C. and a pressure from about 100 kPa to about 3550 kPa (atmospheric to about 500 psig) in the presence of dehydrogenation catalyst. A suitable dehydrogenation catalyst comprises one or more elements or compounds thereof selected from Group 10 of the Periodic Table of Elements, for example, platinum, and/or palladium, on a support, such as silica, alumina or carbon nanotubes. In one embodiment, the Group 10 element (such as platinum) is present in amount from 0.1 to 5 wt % of the catalyst. In some cases, the dehydrogenation catalyst may also include tin or a tin compound to improve the selectivity to the desired methyl-substituted biphenyl product. In one embodiment, the tin is present in amount from 0.05 to 2.5 wt % of the catalyst.

The product of the dehydrogenation reaction comprises a mixture of dimethylbiphenyl isomers together with co-produced hydrogen, and up to 90 wt %, more typically from 0 to 30 wt %, residual (methylcyclohexyl)toluenes. In addition, the dehydrogenation product may contain residual toluene, as well as by-products, such as methylcyclohexane, dimethylcyclohexylbenzene, and C₁₅+ heavy hydrocarbons in addition to the target dimethylbiphenyl isomers. Thus, in some embodiments, prior to recovery of the dimethylbiphenyl isomers, the raw dehydrogenation product is subjected to a rough cut separation to remove at least part of the residues and by-products with significantly different boiling points from the dimethylbiphenyl isomers. For example, the hydrogen by-product can be removed and recycled to the hydroalkylation and/or dehydrogenation steps, while residual toluene and methylcyclohexane by-product can be removed and recycled to the hydroalkylation step. Similarly, part of the heavy (C₁₅+) components can be removed in the rough cut separation and can be recovered for use as a fuel or can be reacted with toluene over a transalkylation catalyst to convert some of the dialkylate to additional (methylcyclohexyl)toluene. A suitable rough cut separation can be achieved by distillation. For example, the H₂ and C₇ components can be stripped from the C₁₂₊ components without reflux.

A similar reaction sequence involving hydroalkylation followed by dehydrogenation can be used with xylene as the starting material, in which case the hydroalkylation step will yield a mixture of (dimethylcyclohexyl)xylene isomers and the dehydrogenation step will produce a mixture of (dimethylphenyl)xylene isomers. In addition, a mixture of toluene and xylene can be used as the hydroalkylation feedstock, in which case the hydroalkylation reaction produces methylcyclohexylbenzenes and/or cyclohexyltoluenes, in addition to cyclohexylbenzene and (methylcyclohexyl)toluenes.

Additionally, or alternately, benzene can be used as a feedstock in the hydroalkylation/dehydrogenation process described above. In this case, the hydroalkylation reaction converts benzene to cyclohexyl benzene (CHB) which is then dehydrogenated to produce biphenyl. The biphenyl can then be reacted with methanol to produce methyl-substituted biphenyl compounds. Any known alkylation catalyst can be used for the methylation reaction, such as an intermediate pore molecular sieve having a Constraint Index (as defined in U.S. Pat. No. 4,016,218) of 3 to 12, for example ZSM-5. The composition of the methylated product will depend on the catalyst and conditions employed in the methylation reaction and the molar ratio of methanol to biphenyl, but will typically comprise a mixture of the different isomers of monomethyl and dimethyl biphenyl compounds.

An alternative process for producing methyl-substituted biphenyl compounds via benzene involves oxidative coupling in which the benzene can be converted directly to biphenyl by reaction with oxygen over an oxidative coupling catalyst as follows:

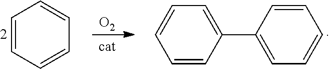

Details of the oxidative coupling of benzene can be found in Ukhopadhyay, Sudip; Rothenberg, Gadi; Gitis, Diana; Sasson, Yoel, Casali Institute of Applied Chemistry, Hebrew University of Jerusalem, Israel, Journal of Organic Chemistry, (2000), 65(10), pp. 3107-3110, incorporated herein by reference. The resultant biphenyl can then be methylated to produce methyl-substituted biphenyl compounds.

### Oxidation of Methyl-Substituted Biphenyl Isomers

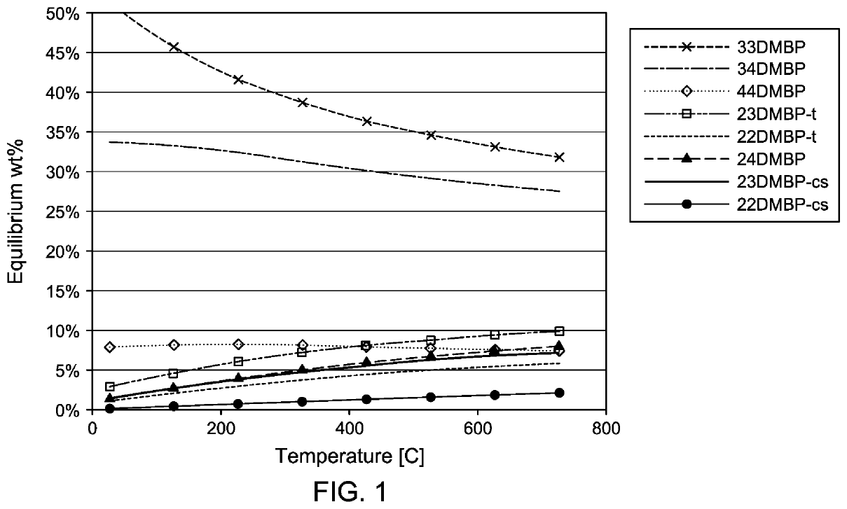

Irrespective of the synthesis process employed methyl-substituted biphenyl compounds and the separation protocol used to remove impurities and residual feedstocks, the product of the reaction sequences described above will comprise a mixture of different isomers of one or more methyl-substituted biphenyl compounds, in which the isomers are at or near their equilibrium distribution. For example, the equilibrium distribution of the different isomers of DMPB over a temperature range from 20 to 750° C. is shown in FIG. 1.

In the process described herein, the composition of the as-synthesized mixture of methyl-substituted biphenyl compounds is adjusted prior to oxidation from the equilibrium isomer distribution to produce an isomer mixture rich in isomer(s) having a methyl group at a 4-position on at least one benzene ring as compared isomer(s) having a methyl group at a 2-position or a 3-position on at least one benzene ring. In this way, it is found to be possible to increase the oxidation rate of the isomer(s) having a methyl group at a 2-position or a 3-position on at least one benzene ring.

Thus, in one embodiment, the isomer mixture fed to the oxidation step comprises a mixture of isomers of at least one methyl-substituted biphenyl compound, wherein the mixture comprises at least 20 wt % of isomer(s) having a methyl group at a 2-position or a 3-position on at least one benzene ring and at least 50 wt %, such as at least 60 wt %, of isomer(s) having a methyl group at a 4-position on at least one benzene ring, wherein the percentages are based on the total weight of the at least one methylbiphenyl compound in the mixture.

For example, where the at least one methyl-substituted biphenyl compound comprises monomethylbiphenyl, suitable mixtures may comprise at least 20 wt %, for example at least 30 wt %, such as at least 40 wt %, for example up to 50 wt %, of isomer(s) having its methyl group at a 2-position or a 3-position on one benzene ring, wherein the percentages are based on the total weight of the at least one methylbiphenyl compound in the mixture. Additionally or alternately, suitable mixtures may comprise at least 50 wt %, such as at least 60 wt %, up to 70 wt % or even up to 80 wt %, of isomer(s) having its methyl group at a 4-position on at least one benzene ring, again wherein the percentages are based on the total weight of the at least monomethylbiphenyl mixture.

Additionally, or alternately, where the at least one methyl-substituted biphenyl compound comprises dimethylbiphenyl, the isomer mixture fed to the oxidation step may comprise at least 20 wt %, for example at least 30 wt %, such as at least 40 wt %, for example, up to 50 wt %, of at least one dimethylbiphenyl isomer selected from the group consisting of 2,3′, and 3,3′ dimethylbiphenyl isomers and comprise at least 50 wt %, such as at least 60 wt %, up to 70 wt % or even up to 80 wt %, of one or more 4,Y′ (where Y′ is 3′ or 4′) dimethylbiphenyl isomers, wherein the percentages are based on the total weight of all dimethylbiphenyl isomers in the mixture. Thus, for example, the isomer mixture may comprise from 20 to 50 wt % of 3,3′ dimethylbiphenyl or from 20 to 50 wt % of 2,3′ dimethylbiphenyl. Generally, the isomer mixture should contain no more than 5 wt %, such as no more than 1 wt %, (methylcyclohexyl)toluene, based on the total weight of the mixture.

Adjusting the isomer distribution of the as-synthesized mixture of methyl-substituted biphenyl compounds to that required for the oxidation step can be at least partially effected by crystallization and/or distillation operating below or, more preferably at, atmospheric pressure. Thus, in the case of dimethylbiphenyl, the normal boiling points and temperatures of fusion of the different isomers are shown in Table 1 below:

TABLE 1

| Isomer | Normal Boiling Point (K) | Fusion Temperature (K) |
|---|---|---|
| 2,2′ | 531 | 320 |
| 2,3′ | 546 | |
| 2,4′ | 554 | 218 |
| 3,3′ | 559 | 278 |
| 3,4′ | 569 | 283 |
| 4,4′ | 568 | 394 |

Additionally, or alternately, adjustment of the isomer distribution of the as-synthesized mixture of methyl-substituted biphenyl compounds can be at least partially effected by isomerization.

Oxidation of the resultant mixture of methyl-substituted biphenyl compounds can be performed by any process known in the art, such as by reacting the methyl-substituted biphenyl compounds with an oxidant, such as oxygen, ozone or air, or any other oxygen source, such as hydrogen peroxide, in the presence of a catalyst and with or without a promoter, such as Br compound, at temperatures from 30° C. to 300° C., such as from 60° C. to 200° C. Suitable catalysts comprise Co or Mn or a combination of both metals. Where the oxidation is performed in a reactor, the oxygen concentration in the gas effluent is preferably greater than about 5 mol %, more preferably greater than about 10 mol %. The oxidation converts at least one methyl group on the methyl-substituted biphenyl compound to the associated carboxylic acid. Thus, in the case of dimethylbiphenyl, the oxidation product can comprise methyl biphenyl carboxylic acid, biphenyl dicarboxylic acid, or a mixture thereof. The resultant carboxylic acids can then be reacted with alcohols to produce esters and polyesters.

For example, biphenyl carboxylic acids can be esterified with one or more C₁ to C₁₆ alcohols under conditions including temperatures of 0-300° C. and the presence or absence of homogeneous or heterogeneous esterification catalysts, such as Lewis or Bronsted acid catalysts. Suitable alcohols are “oxo-alcohols,” by which is meant an organic alcohol, or mixture of organic alcohols, which is prepared by hydroformylating an olefin, followed by hydrogenation to form the alcohols. Typically, the olefin is formed by light olefin oligomerization over heterogeneous acid catalysts, which olefins are readily available from refinery processing operations. The reaction results in mixtures of longer-chain, branched olefins, which subsequently form longer chain, branched alcohols, as described in U.S. Pat. No. 6,274,756, incorporated herein by reference in its entirety. Another source of olefins used in the OXO process are through the oligomerization of ethylene, producing mixtures of predominately straight chain alcohols with lesser amounts of lightly branched alcohols.

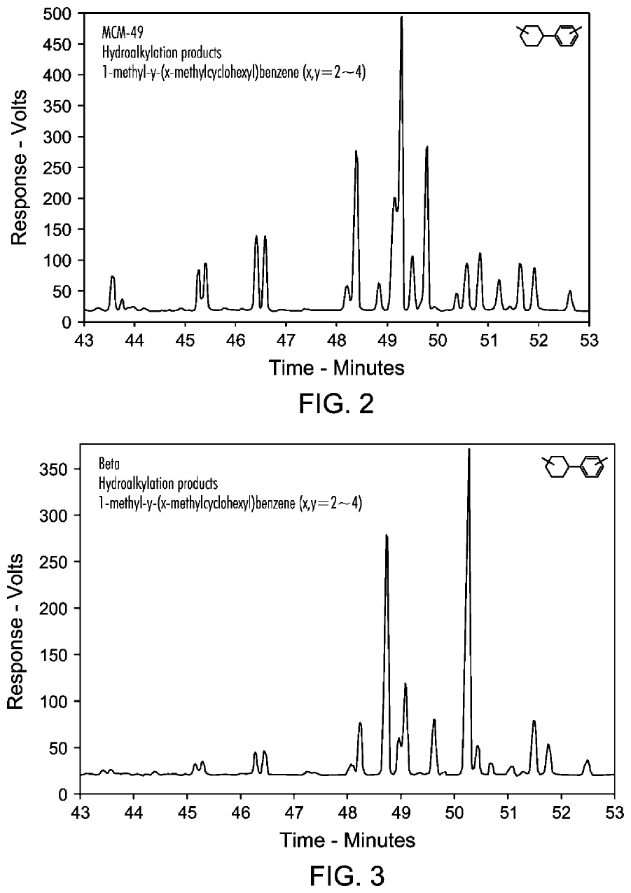

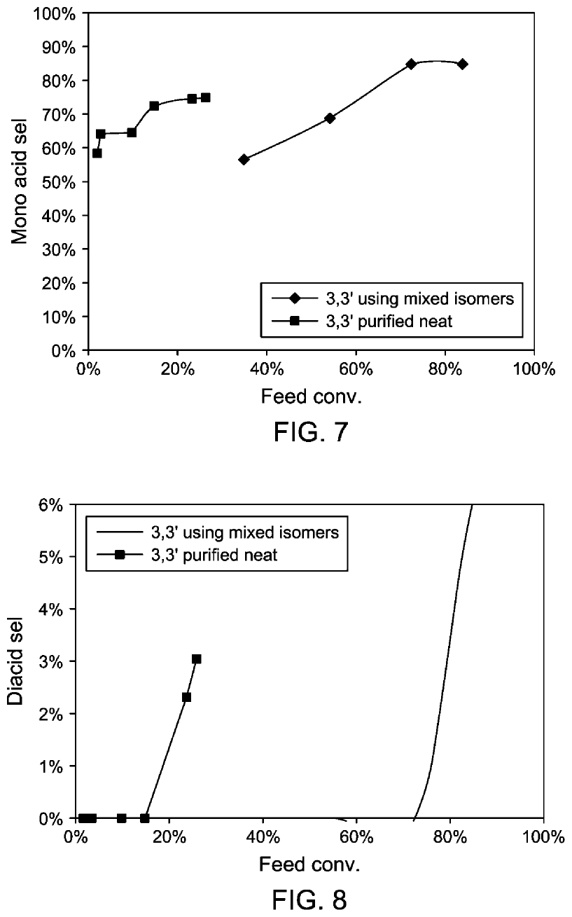

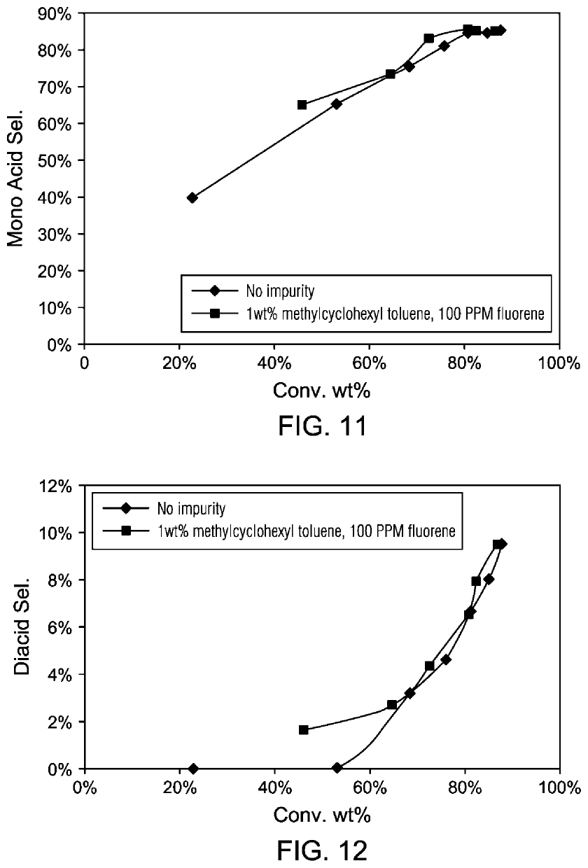

The invention will now be more particularly described with reference to the following non-limiting Examples and FIGS. 2 to 12 of the accompanying drawings. In the Examples, all parts and percentages are by weight unless otherwise indicated. Room temperature is 23° C. unless otherwise indicated.

### Example 1: Synthesis of 0.3% Pd/MCM-49 Hydroalkylation Catalyst

80 parts MCM-49 zeolite crystals are combined with 20 parts pseudoboehmite alumina, on a calcined dry weight basis. The MCM-49 and pseudoboehmite alumina dry powder is placed in a muller and mixed for about 10 to 30 minutes. Sufficient water and 0.05% polyvinyl alcohol is added to the MCM-49 and alumina during the mixing process to produce an extrudable paste. The extrudable paste is formed into a 1/20 inch (0.13 cm) quadrulobe extrudate using an extruder and the resulting extrudate is dried at a temperature ranging from 250° F. to 325° F. (120° C. to 163° C.). After drying, the dried extrudate is heated to 1000° F. (538° C.) under flowing nitrogen. The extrudate is then cooled to ambient temperature and humidified with saturated air or steam.

After the humidification, the extrudate is ion exchanged with 0.5 to 1 N ammonium nitrate solution. The ammonium nitrate solution ion exchange is repeated. The ammonium nitrate exchanged extrudate is then washed with deionized water to remove residual nitrate prior to calcination in air. After washing the wet extrudate, it is dried. The exchanged and dried extrudate is then calcined in a nitrogen/air mixture to a temperature 1000° F. (538° C.). Afterwards, the calcined extrudate is cooled to room temperature. The 80% MCM-49, 20% Al₂O₃ extrudate was incipient wetness impregnated with a palladium (II) chloride solution (target: 0.30% Pd) and then dried overnight at 121° C. The dried catalyst was calcined in air at the following conditions: 5 volumes air per volume catalyst per minute, ramp from ambient to 538° C. at 1° C./min and hold for 3 hours.

### Example 2: Synthesis of 0.3% Pd/Beta Hydroalkylation Catalyst

80 parts beta zeolite crystals are combined with 20 parts pseudoboehmite alumina, on a calcined dry weight basis. The beta and pseudoboehmite are mixed in a muller for about 15 to 60 minutes. Sufficient water and 1.0% nitric acid is added during the mixing process to produce an extrudable paste. The extrudable paste is formed into a 1/20 inch quadrulobe extrudate using an extruder. After extrusion, the 1/20th inch quadrulobe extrudate is dried at a temperature ranging from 250° F. to 325° F. (120° C. to 163° C.). After drying, the dried extrudate is heated to 100° F. (538° C.) under flowing nitrogen and then calcined in air at a temperature of 100° F. (538° C.). Afterwards, the calcined extrudate is cooled to room temperature. The 80% Beta, 20% Al₂O₃ extrudate was incipient wetness impregnated with a tetraammine palladium (II) nitrate solution (target: 0.30% Pd) and then dried overnight at 121° C. The dried catalyst was calcined in air at the following conditions: 5 volumes air per volume catalyst per minute, ramp from ambient to 538° C. at 1° C./min and hold for 3 hours.

### Example 3: Hydroalkylation Catalyst Testing

Each of the catalysts of Example 1 and 2 was tested in the hydroalkylation of a toluene feed using the reactor and process described below. The reactor comprised a stainless steel tube having an outside diameter of: ⅜ inch (0.95 cm), a length of 20.5 inch (52 cm) and a wall thickness of 0.35 inch (0.9 cm). A piece of stainless steel tubing having a length of 8% inch (22 cm) and an outside diameter of: ⅜ inch (0.95 cm) and a similar length of 4 inch (0.6 cm) tubing was used in the bottom of the reactor (one inside of the other) as a spacer to position and support the catalyst in the isothermal zone of a furnace. A ¼ inch (0.6 cm) plug of glass wool was placed on top of the spacer to keep the catalyst in place. A ⅛ inch (0.3 cm) stainless steel thermo-well was placed in the catalyst bed to monitor temperature throughout the catalyst bed using a movable thermocouple.

The catalyst was sized to 20/40 sieve mesh or cut to 1:1 length to diameter ratio, dispersed with quartz chips (20/40 mesh) then loaded into the reactor from the top to a volume of 5.5 cc. The catalyst bed typically was 15 cm. in length. The remaining void space at the top of the reactor was filled with quartz chips, with a ¼ plug of glass wool placed on top of the catalyst bed being used to separate quartz chips from the catalyst. The reactor was installed in the furnace with the catalyst bed in the middle of the furnace at a pre-marked isothermal zone. The reactor was then pressure and leak tested typically at 300 psig (2170 kPa).

The catalyst was pre-conditioned in situ by heating to 25° C. to 240° C. with H₂ flow at 100 cc/min and holding for 12 hours. A 500 cc ISCO syringe pump was used to introduce chemical grade toluene feed to the reactor. The feed was pumped through a vaporizer before flowing through heated lines to the reactor. A Brooks mass flow controller was used to set the hydrogen flow rate. A Grove “Mity Mite™” back pressure controller was used to control the reactor pressure typically at 150 psig (1135 kPa). Gas Chromatograph (GC) analyses were taken to verify feed composition. The feed was then pumped through the catalyst bed held at the reaction temperature of 120° C. to 180° C. at a WHSV of 2 and a pressure of 15-200 psig (204-1480 kPa). The liquid products exiting the reactor flowed through heated lines routed to two collection pots in series, the first pot being heated to 60° C. and the second pot cooled with chilled coolant to about 10° C. Material balances were taken at 12 to 24 hr. intervals. Samples were taken and diluted with 50% ethanol for analysis. An Agilent 7890™ gas chromatograph with FID detector was used for the analysis. The non-condensable gas products were routed to an on line HP 5890™ GC

The results of the hydroalkylation testing are shown in FIGS. 2 and 3 and in Tables 2 and 3.

TABLE 2

| | MCM-49 HA product | Beta HA product |
|---|---|---|
| y-(x-methylcyclohexyl)toluene (x, y = 2, 3, 4) | 89.29% | 39.82% |
| y-(1-methylcyclohexyl)toluene (y = 2, 4) | 3.03% | 53.26% |

TABLE 3

| | | | | Selectivity to | Selectivity to |
|---|---|---|---|---|---|
| | | | Toluene | methyl- | dimethyl |
| Example | Catalyst | | conversion | cyclohexane | bi(cyclohexane) |
| 1 | 0.3% | Pd-MCM49 | 37% | 23% | 1.40% |
| 2 | 0.3% | Pd/Beta | 40% | 65% | 1.60% |

Table 2 clearly shows that the MCM-49 catalyst can provide much higher amounts of the desired hydroalkylation products (y-(x-methylcyclohexyl)toluene (x,y=2,3,4)) than the zeolite beta catalyst, and a much lower amount of undesired y-(1-methylcyclohexyl)toluene (y=2, 4).

As can be seen from Table 3, the Pd/MCM-49 catalyst has much lower selectivity towards the production of the fully saturated by-products, methylcyclohexane and dimethylbi(cyclohexane), than Pd/beta.

### Example 4: Production of 1% Pt/0.15% Sn/SiO2 Dehydrogenation Catalyst

A 1% Pt/0.15% Sn/SiO2 catalyst was prepared by incipient wetness impregnation, in which a 1/20″ (1.2 mm) quadrulobe silica extrudate was initially impregnated with an aqueous solution of tin chloride and then dried in air at 121° C. The resultant tin-containing extrudates were then impregnated with an aqueous solution of tetraammine Pt nitrate and again dried in air at 121° C. The resultant product was calcined in air at 350° C. for 3 hours before being used in subsequent catalyst testing.

### Example 5: Dehydrogenation Catalyst Testing

The catalyst of Example 4 was used to perform dehydrogenation testing on part of the effluent of each hydroalkylation reaction of Example 3. The same reactor and testing protocol as described in Example 3 were used to perform dehydrogenation tests, except the dehydrogenation catalyst was pre-conditioned in situ by heating to 375° C. to 460° C. with H₂ flow at 100 cc/min and holding for 2 hours. In addition, in the dehydrogenation tests the catalyst bed was held at the reaction temperature of 425° C. at a WHSV of 2 and a pressure of 100 psig (790 kPa). The tests were also repeated with a commercial 0.3 wt % Pt/A203 dehydrogenation catalyst supplied by Akzo.

The analysis is done on an Agilent 7890™ GC with 150 vial sample tray set up as follows:
Inlet Temp: 220° C. Detector Temp: 240° C. (Col+make up=constant);
Temp Program: Initial temp 120° C. hold for 15 min., ramp at 2° C./min to 180° C., hold 15 min; ramp at 3° C./min. to 220° C. and hold till end. Column Flow: 2.25 ml/min. (27 cm/sec); Split mode, Split ratio 100:1;
Injector: Auto sampler (0.2 μl) Column Parameters;
Two columns joined to make 120 Meters (coupled with Agilent ultimate union, deactivated; and
Column #Front end: Supelco β-Dex 120; 60 m×0.25 mm×0.25 μm film joined to Column #2 back end: γ-Dex 325: 60 m×0.25 mm×0.25 m film.

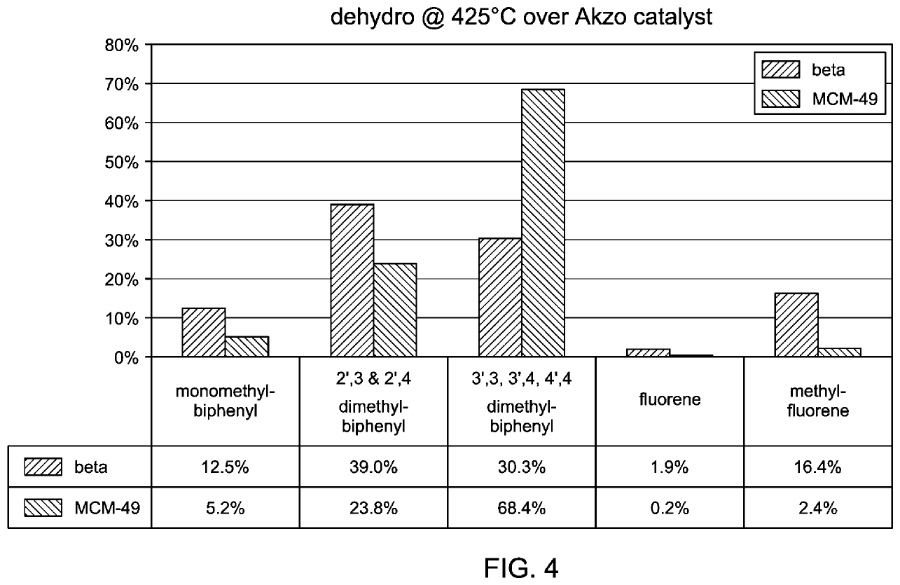

The results of the dehydrogenation testing with the catalyst of Example 4 are shown in Table 4 and FIG. 4 and with the commercial Akzo catalyst are shown in Table 4.

TABLE 4
Dehydro @ 425° C. over 1% Pt/0.15Sn/SiO2 catalyst

| | MCM-49 HA product over | Beta HA product over selective |
|---|---|---|
| | selective dehydrogenation catalyst | dehydrogenation catalyst |
| 3-methyl biphenyl | 0.70% | 2.47% |
| 4-methyl biphenyl | 0.79% | 4.98% |
| 2,2′ dimethyl biphenyl | 1.21% | 1.04% |
| 2,3′ dimethyl biphenyl | 9.52% | 8.42% |
| 2,4′ dimethyl biphenyl | 13.14% | 12.64% |
| 3,3′ dimethyl biphenyl | 15.98% | 13.21% |
| 3,4′ dimethyl biphenyl | 39.64% | 36.26% |
| 4,4′ dimethyl biphenyl | 18.76% | 18.19% |
| fluorene | 0.00% | 0.93% |
| methyl fluorenes | 0.26% | 1.87% |

### Example 6: Oxidation of Mixed DMBP Isomers

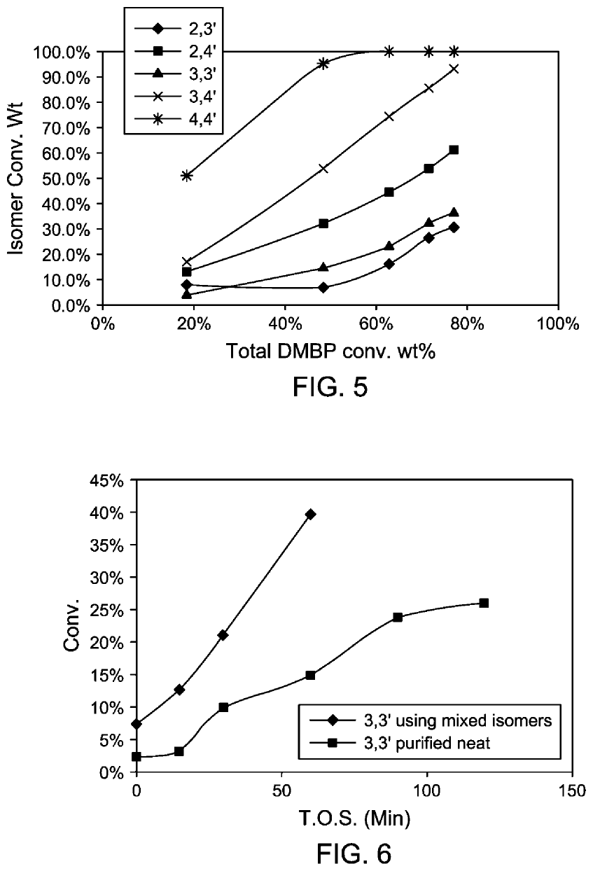

Oxidation was done batchwise. A 300 ml Parr reactor was charged with 30 grams of a dimethylbiphenyl mixed isomer feed (with the following composition 0.6% 2,3′-DMBP, 1.9% 2,4′-DMBP, 29% 3,3′-DMBP, 52.8% 3,4′-DMBP, and 15.7% 4,4′-DMBP), 120 gms acetic acid, and 1500 ppm Co acetate. The reactor was sealed and pressurized to 500 psig (3549 kPa-a) with nitrogen. The reactor was heated to 150° C. with a stir rate of 1200 rpm under 1500 cc/min N₂. When the temperature reached 150° C., N₂ was switched to air at the same flow rate. During the reaction, liquid samples were taken for GC analysis and the oxygen concentration in the gas effluent was measured. After 2 hours reaction time the air flow was switched to N₂, the reactor was cooled to room temperature then depressurized. The oxidation conversion/selectivity profile is shown in FIG. 5.

### Example 7: Oxidation of 3,3′-DMBP

Oxidation was done batchwise. A 300 ml Parr reactor was charged with 30 grams of 3,3′-DMBP, 120 gms acetic acid, 1500 ppm Co acetate. The reactor was sealed and pressurized to 500 psig (3549 kPa-a) with nitrogen. The reactor was heated to 150° C. with a stir rate of 1200 rpm under 1500 cc/min N₂. When the temperature reached 150° C., N₂ was switched to air at the same flow rate. During the reaction liquid samples were taken for GC analysis and the oxygen concentration in the gas effluent was measured. After 2 hours reaction time the air flow was switched to N₂, the reactor was cooled to room temperature then depressurized. The oxidation conversion/selectivity profile for the pure 3,3′-DMBP of this Example is compared with that for the 3,3′-DMBP in the DMBP isomer mixture of Example 6 in FIGS. 6 to 8.

### Example 8: Oxidation of 2,3′-DMPB

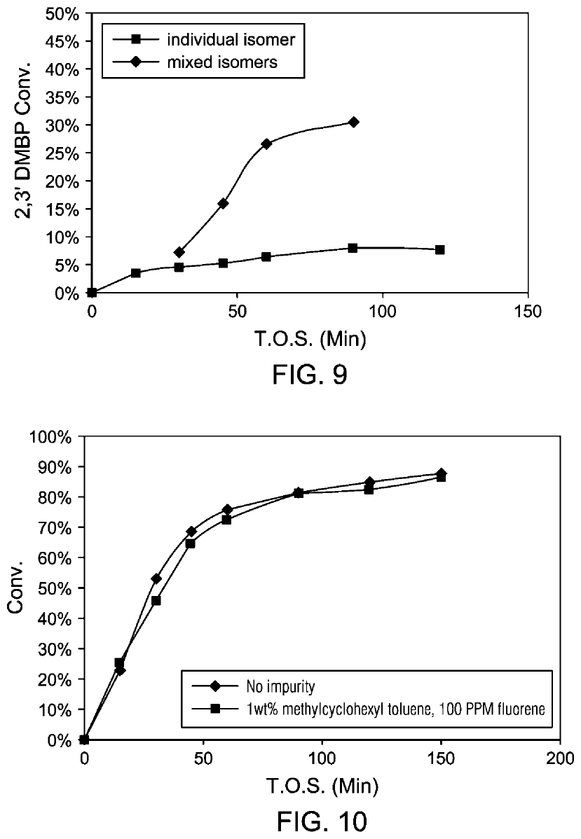

Oxidation was done batchwise. A 300 ml Parr reactor was charged with 30 grams of 2,3′-DMBP, 120 gms acetic acid, 1500 ppm Co acetate. The reactor was sealed and pressurized to 500 psig (3549 kPa-a) with nitrogen. The reactor was heated to 150° C. with a stir rate of 1200 rpm under 1500 cc/min N₂. When the temperature reached 150° C., N₂ was switched to air at the same flow rate. During the reaction liquid samples were taken for GC analysis and the oxygen concentration in the gas effluent was measured. After 2 hours reaction time the air flow was switched to N₂, the reactor was cooled to room temperature then depressurized. The oxidation conversion/selectivity profile for the pure 2,3′-DMBP of this Example is compared with that for the 2,3′-DMPB in the DMPB isomer mixture of Example 6 in FIG. 9.

The data in FIGS. 6 to 9 clearly indicate that the composition of the feed is very critical for obtaining higher conversion rate and better selectivity towards mono acid. These were achieved by preparing a feed which contains higher concentration of 4,X′(x=3,4) which are highly active in generating free radicals and maintaining a high steady state concentration of radicals. Also, this allows the low activity isomers (e.g., 3,3′ and 2,X′ (X=3,4)) to be converted at a faster oxidation rate.

### Example 9: Oxidation of Mixed DMBP Isomers Spiked with 1 wt % Methylcyclohexyl Toluene and 100 PPM Fluorene

Oxidation was done batchwise. A 300 ml Parr reactor was charged with 30 grams of a dimethylbiphenyl mixed isomer feed (with the following composition 0.6% 2,3′-DMBP, 1.9% 2,4′-DMBP, 29% 3,3′-DMBP, 52.8% 3,4′-DMBP, and 15.7% 4,4′-DMBP) spiked with 1 wt % methylcyclohexyl toluene and 100 PPM fluorene, 120 gms acetic acid, and 1500 ppm Co acetate. The reactor was sealed and pressurized to 500 psig (3549 kPa-a) with nitrogen. The reactor was heated to 150° C. with a stir rate of 1200 rpm under 1500 cc/min N₂. When the temperature reached 150° C., N₂ was switched to air at the same flow rate. During the reaction liquid samples were taken for GC analysis and the oxygen concentration in the gas effluent was measured. After 2 hours reaction time the air flow was switched to N₂, the reactor was cooled to room temperature then depressurized. The oxidation conversion/selectivity profile for the spiked DMBP mixture of this Example is compared with that for unspiked DMPB isomer mixture of Example 6 in FIGS. 10 to 12.

The data in FIGS. 10 to 12 show that the oxidation rate and acid selectivity are substantially unchanged when the mixed DMBP feed is contaminated with methylcyclohexyltoluene and/or fluorene, which inhibit free radical formation via a beta scission mechanism. Higher concentration of 4,X′ isomers increases the initiation reaction rate and decreases the termination reaction rates, improving the overall oxidation rate.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention. All documents described herein are incorporated by reference herein, including any priority documents and/or testing procedures to the extent they are not inconsistent with this text. Likewise, the term “comprising” is considered synonymous with the term “including” and whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

1-15. (canceled)

16. A process for producing methyl biphenyl carboxylic acid and/or biphenyl dicarboxylic acid, the process comprising:
(a3) contacting a feed comprising benzene with hydrogen in the presence of a hydroalkylation catalyst under conditions effective to produce a hydroalkylation reaction product comprising cyclohexylbenzenes;
(b3) dehydrogenating at least part of the hydroalkylation reaction product in the presence of a dehydrogenation catalyst under conditions effective to produce a dehydrogenation reaction product comprising biphenyl;
(c3) reacting at least part of the dehydrogenation reaction product with a methylating agent in the presence of an alkylation catalyst under conditions effective to produce a methylation reaction product comprising methyl-substituted biphenyl compounds;
(d3) adjusting the composition of at least part of the methylation reaction product to produce a mixture comprising isomers of at least one methyl-substituted biphenyl compound, wherein the mixture comprises at least 20 wt % of isomer(s) having a methyl group at a 2-position or a 3-position on at least one benzene ring and at least 50 wt % of isomer(s) having a methyl group at a 4-position on at least one benzene ring, wherein said percentages are based on the total weight of the at least one methylbiphenyl compound in the mixture; and
(e3) oxidizing the mixture produced in (d3).

17. The process of claim 16, wherein the hydroalkylation catalyst comprises an acidic component and a hydrogenation component.

18. The process of claim 17, wherein the acidic component of the hydroalkylation catalyst comprises a molecular sieve of the MCM-22 family.

19. The process of claim 16, wherein the adjusting comprises at least one of distillation, crystallization and isomerization.

20-25. (canceled)
